(19) United States
(12) Patent Application Publication
(10) Pub. No.: US 2020/0338319 A1
(43) Pub. Date: Oct. 29, 2020

(54) DEVICE FOR DELIVERING ENERGY AND/OR MEASURING ELECTRICAL ACTIVITY

(71) Applicant: BIOTRONIK AG, Buelach (CH)

(72) Inventors: Chritian BLUEMEL, Kuesnacht (CH); Erik TRIP, Singapore (SG); Jan Helge RICHTER, Berlin (DE); Marc Steffen SCHURR, Berlin (DE)

(73) Assignee: Biotronik AG, Buelach (CH)

(21) Appl. No.: 16/092,227

(22) PCT Filed: Mar. 27, 2017
(86) PCT No.: PCT/EP2017/057207

§ 371 Date: Oct. 9, 2018

(30) Foreign Application Priority Data
Apr. 8, 2016 (DE) 10 2016 106 478.5

(51) Int. Cl. A61M 25/10; A61N 1/05; A61B 18/14; A61B 5/00

(52) U.S. Cl. CPC A61M 25/1002; A61N 1/05; A61B 2018/00023; A61B 5/6853; A61B 18/1492

(57) ABSTRACT

A device for delivering energy and/or measuring electrical activity in a tubular part of a body has a catheter and/or an inflatable balloon and at least one electrode. A distal end of the catheter with the at least one electrode, or the at least one electrode, is disposed on the outside of an envelope of the balloon. So as to achieve good contact between the at least one electrode and the wall of the vessel to be treated and continuous blood flow, the elasticity of the envelope of the balloon is less in at least one first region than in a second region that different from the first region. The distal end of the catheter with the at least one electrode is disposed in the at least one first region, or the at least one electrode is disposed in the at least one second region of the balloon.

The present invention relates to a device for delivering energy and/or measuring electrical activity in a tubular part of a body, for example a blood vessel or a tubular organ, such as ureter, intestine, trachea, renal pelvis, esophagus, ENT tract, comprising a catheter and/or a balloon and at least one electrode.

Intravascular catheters, especially for applications in the heart/thorax region, comprising an active or a passive electrode are already known, which can be introduced into primary veins or arteries, such as the femoral vein, and advanced from there to various locations of the heart or to the coronary vessels. These catheters are used to represent or stimulate the electrical activity of the heart or remove regions exhibiting abnormal electrical activity. The latter, which is the ablation therapy, is used as a treatment for cardiac dysrhythmia, for example.

Furthermore, catheters are used today to achieve a reduction in blood pressure by way of neuromodulation of the renal nerves (renal plexus). This treatment is based on the assumption that the sympathetic nerve strand located near the renal artery influences blood pressure. It was found that partial traumatization of this nerve causes a sustained decrease in blood pressure, and that this treatment is promising in particular for treatment refractory patients who do not respond, or no longer respond, to conventional pharmacological approaches.

By way of known devices for delivering energy and/or measuring electrical activity which are based on a catheter, it is very difficult to achieve reliable contact between the electrodes of the catheter and the vascular wall. Moreover, checking the wall contact prior to denervation by way of X-rays or electrical measurements is only possible with low accuracy.

To achieve this object, an inflatable balloon can be used, as is described in the published prior art U.S. Pat. No. 8,548,600 B2, for example, so as to displace the distal end of the catheter of the electrode, which is disposed in a helical shape on the outside of the balloon, in the direction of the vascular inner wall and to be affixed there. The balloons used for this purpose, which are cylindrical when inflated, however, tend to completely close the treated blood vessel, thereby interrupting the blood flow to the organs located behind of the treated site (in the renal artery, for example, to the kidney), which is not desirable. The occlusion of the vessel has the added disadvantage that the intima of the vessel cannot be cooled by the blood flow in the vessel during the procedure. This damages the intima in a more extensive area.

The object is therefore to create a device that effects reliable contact between the electrode and the vascular wall, while also avoiding the above-described disadvantages.

The above object is achieved by a device in which the elasticity (extensibility) of the envelope of the balloon is less in at least one first region or section than in at least one second region or section different from the first region, wherein the distal end of the catheter comprising the at least one electrode is disposed in the at least one first region.

The above object is alternatively achieved by a device comprising an inflatable balloon and at least one electrode, wherein the at least one electrode is disposed on the outside of an envelope of the balloon, wherein the elasticity of the envelope of the balloon is less in at least one first region than in at least one second region different from the first region, wherein the at least one electrode is disposed in the at least one second region.

The difference in the elasticity between the first region and the second region is designed in such a way here that a difference in height of the envelope of the balloon, when expanded, between the two regions in the radial direction, based on the balloon envelope, is at least 0.5 mm, and preferably in the range between 1 mm and 2.5 mm, so that, for example, the blood flow is not interrupted, or is not entirely interrupted, in this location.

This is achieved, for example, by using materials that have differing moduli of elasticity (elastic moduli) in the first region and the second region for the envelope of the balloon, which is to say a first material is used for the first region of the envelope of the balloon, and a second material that is different from the first material and has a lower modulus of elasticity is used for the second region. [The elastic modulus is an imaginary stress that would elastically extend a sample rod to twice the length (which is to say strain ε=1).] The lower the modulus of elasticity of the material, the more pronounced is the change in longitude thereof at the same stress or, during the inflation of a balloon, at the same pressure, as compared to a material having a higher modulus of elasticity.

The materials listed in the table below can be used for the first or second region of the balloon.

| | | Typical modulus of | Additional |
|---|---|---|---|
| Material | | elasticity in | information/trade |
| abbreviation | Material designation | [N/mm²] | name |
| 1.4310 | Stainless steel | 185000 | V2a spring hard |
| PEEK | Polyether ether ketone | 4400 | |
| PPTA | Poly(p-phenylene | 59000 | Aramide, Keflar |
| | terephthalamide) | | |
| PI | Polyimide | 2500 | Kapton |
| PMPI | Poly(m-phenylene | 17000 | Nomex, |
| | terephthalamide) | | Teijinconex |
| PA | Polyamide | 1500 | |
| PET | Polyethylene | 3000 | |
| | terephthalate | | |
| PE | Polyethylene | 1000 | PE-LD |
| PU | Polyurethane | 100 | Shore 80A |
| NR | Natural rubber | 10 | Latex |
| Si | Silicon | 80 | |
| SBR | Styrene-butadiene | 10 | Rubber |
| | rubber | | |

For example, use of the material pairing PI and PU results in a difference in the change of longitude at an approximate 25:1 ratio.

The same material used together with a simple wall thickness for a second region and together with double the wall thickness for a first region still results in a difference in the change of longitude at an approximately 2:1 ratio, with other ratios remaining the same.

The balloon of the device according to the invention comprising a first region and a second region of the balloon envelope, in which the regions have differing strain values, forms an irregular shape when inflated. Due to the lesser elasticity, the first region is not extended as much when the balloon is fully inflated and, consequently, recedes in relation to the second region, which is to say the second region protrudes further, whereby the above-mentioned difference in height is created. As a result, the surface of the inflated balloon is not smooth, but has a relief comprising protruding regions (second regions) and recessed regions (first regions), as well as transition regions therebetween, so that clearances arise for the blood flow or the flow of other body fluids, and the vessel is not entirely closed. In this way, the blood flow in the vessel is not interrupted. In addition to the consistent supply of blood to nearby organs, this has the advantage that the tissue at the denervation or ablation sites continues to be cooled by the blood flow. In this way, deeper lesions can be produced.

Moreover, by disposing the distal end of the catheter comprising the at least one electrode in the at least one first region, secure wall contact of the electrode is established and, consequently, a reliable denervation or ablation result can be achieved. This is accomplished in that the inflated balloon presses the distal end of the catheter against the vascular wall, thereby fixing the position thereof. Furthermore, it was found that pressing the catheter against the vascular wall considerably improves the electrical contact between the catheter and the vessel, and higher electrical conductivity is achieved. Moreover, disposing the distal end of the catheter in the first region having the lower elasticity prevents strain/overextension of the catheter. In the alternative embodiment without catheter, in which the electrodes are provided on the outside of the balloon envelope (and, if necessary, at least partially in the balloon envelope), the secure wall contact is achieved by disposing the at least one electrode in the at least one second region.

In a preferred embodiment of the invention, the envelope of the balloon comprises fibers in the at least one first region, which limit the elasticity of the envelope of the balloon in this region. The elasticity of the fibers disposed in this region is less than that of the envelope of the balloon in the second region. The lesser elasticity in the first region is particularly easy to implement by embedding fibers that have accordingly little elasticity in the volume of the balloon envelope or on the surface thereof. The fibers can be embedded in the balloon envelope by way of an incremental build-up. For example, fibers can be woven onto an inner tube, over which an outer tube is then extruded. On the outside, the fibers can be joined to the balloon envelope by way of adhesive bonding or welding, for example. The fibers of the first region preferably comprise a material or multiple materials from the group consisting of PEEK, PPTA, PI, PA and metals, all materials preferably taking on the form of a ribbon, rope, cord or wire. The balloon envelope can comprise a material or multiple materials from the group consisting of NR, SI, PET, PE and PU, for example.

In another embodiment, the wall thickness of the balloon envelope can be varied so as to implement differing extensibilities, wherein the wall thickness in the at least one first region is greater than the wall thickness in the at least one second region, wherein the wall thicknesses can preferably be varied between 0.01 mm and 0.3 mm. It is advantageous when the wall thickness in the first region is greater than the wall thickness in the second region by at least a factor of 3. Accordingly, it is advantageous when the wall thickness in the first region ranges between 0.01 mm and 0.1 mm, and in the second region ranges between 0.03 mm and 0.3 mm.

So as to achieve a simple arrangement of the distal end of the catheter comprising the electrodes on the surface of the balloon, it is advantageous when the first region has a rhombus shape or when the first region extends in the direction of a longitudinal axis of the balloon or in a spiral shape (helically) about the longitudinal axis of the balloon. The rhombus shape is achieved by mutually intersecting regions extending obliquely with respect to the longitudinal axis in such a way that a second region is disposed within the rhombus outline. Due to the rhombus shape of the first regions, a protuberance- or pillow-like surface relief of the balloon is created when inflated, which yields the above-described clearances for maintaining the blood flow.

A secure arrangement of the catheter comprising the electrodes on the balloon is achieved when the distal region of the catheter is fixed in points on the envelope of the balloon. This can be implemented, for example, by way of a weld or adhesive joint.

In an advantageous embodiment, the at least one electrode disposed on the catheter is designed as a conductive ring or a conductive half shell.

In another embodiment, the catheter can be implemented as a guide catheter (if necessary, comprising an additional lumen for a guide wire). When using the balloon variants shown here, it is also possible, alternatively, to use a stent, and in contrast to known systems, the blood flow is consistently preserved when using such a balloon.

In a further exemplary embodiment of the variant comprising the catheter, the balloon is additionally designed to be electrically active. In this case, at least one electrode is disposed in or on the balloon envelope in the at least one second region and, when inflated, is pushed against the vascular wall, whereby good contact with the vascular wall is established.

Further objectives, features, advantages, and application possibilities of the invention will be apparent from the following description of exemplary embodiments based on the figures. All features described and/or illustrated, either alone or in any arbitrary combination, form the subject matter of the present invention, including independently of their combination in the individual claims or their dependency reference.

In the schematic drawings:

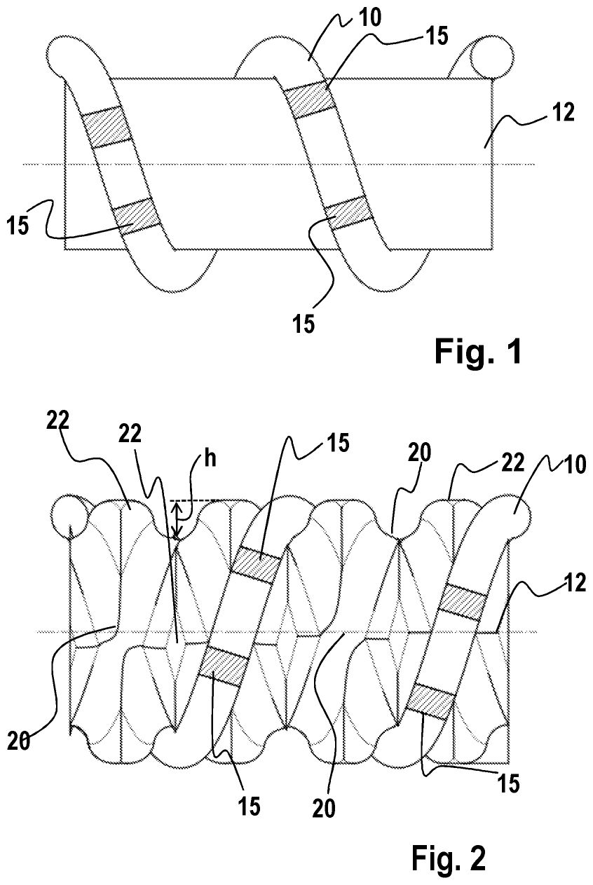

FIG. 1 shows a first exemplary embodiment of a device according to the invention in a first state of the balloon where it is little inflated, in a view from the side;

FIG. 2 shows the exemplary embodiment according to FIG. 1 in a second state of the balloon where it is more strongly inflated, in a view from the side;

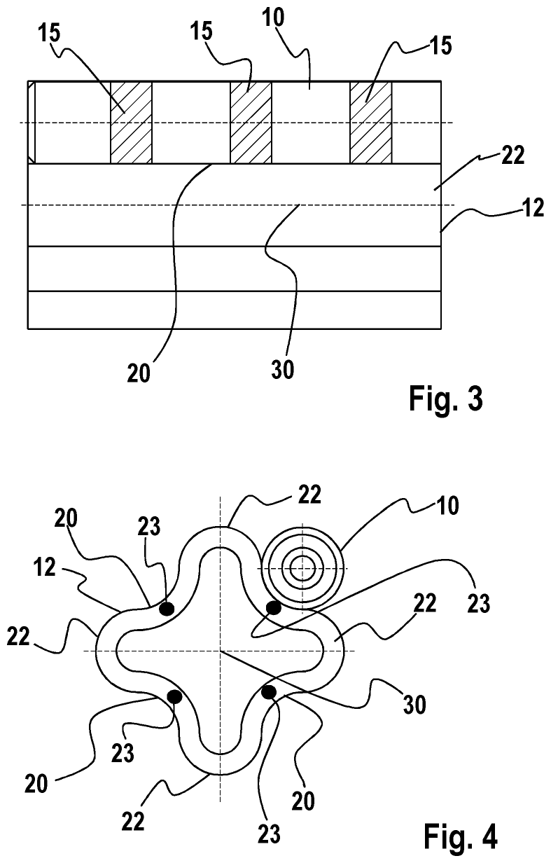

FIG. 3 shows a second exemplary embodiment of a device according to the invention in a more strongly inflated state and in a view from the side;

FIG. 4 shows the exemplary embodiment and the state according to FIG. 3 in a cross-section;

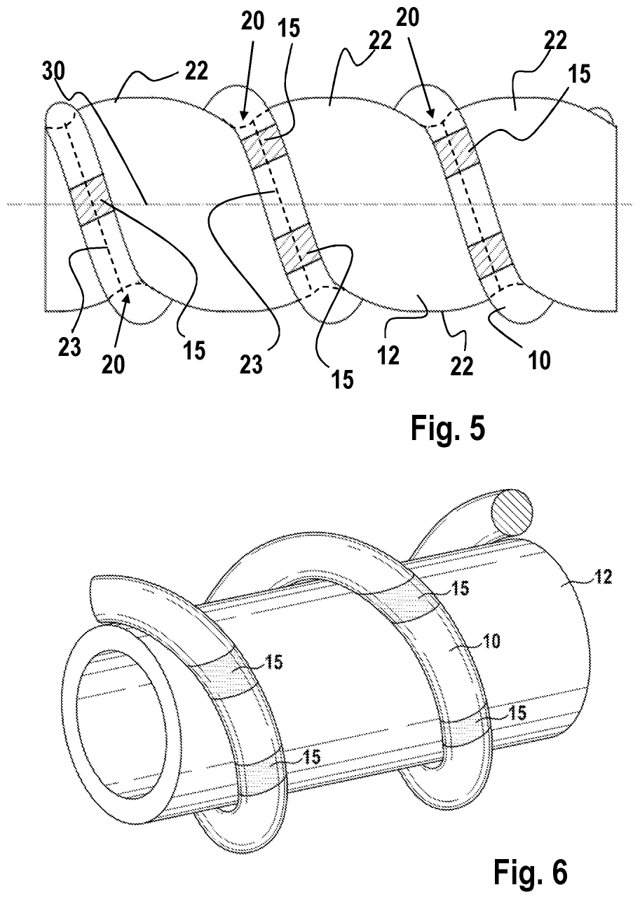

FIG. 5 shows a third exemplary embodiment of a device according to the invention in a more strongly inflated state and in a view from the side;

FIG. 6 shows the exemplary embodiment according to FIGS. 1 and 2 in the first, little inflated state, in a perspective view from the side;

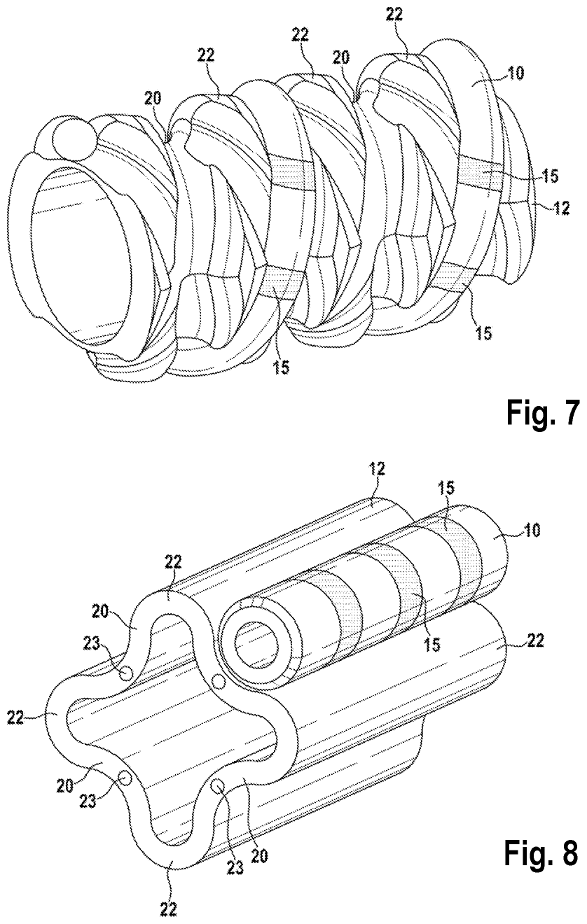

FIG. 7 shows the exemplary embodiment according to FIGS. 1 and 2 in the second, more strongly inflated state, in a perspective view from the side;

FIG. 8 shows the exemplary embodiment and the state according to FIGS. 3 and 4 in a perspective view from the side;

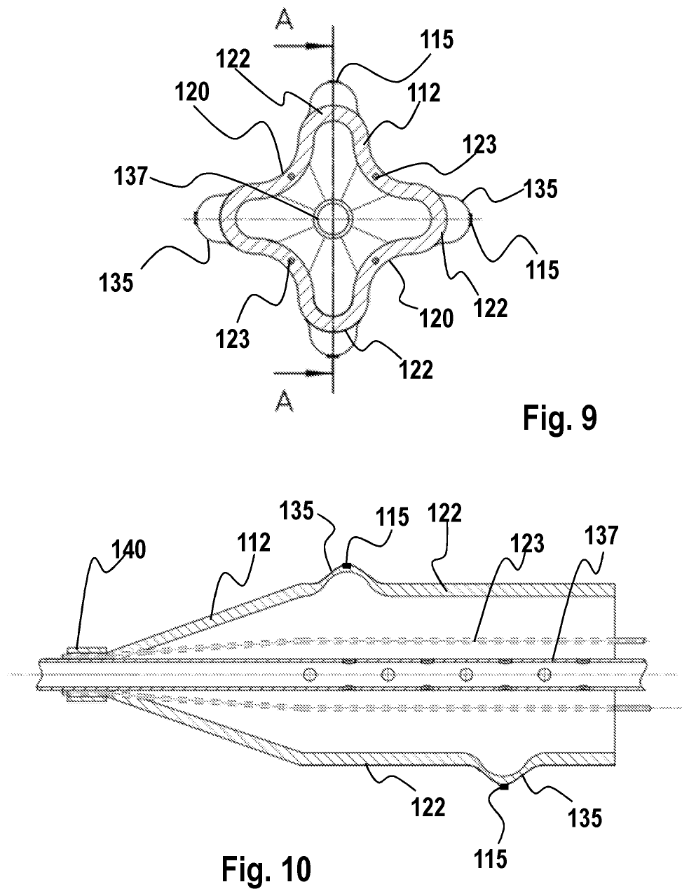

FIG. 9 shows a fourth exemplary embodiment of a device according to the invention in a second, more strongly inflated state in a cross-section;

FIG. 10 shows the exemplary embodiment according to FIG. 9 in the state according to FIG. 9 in a section along the line A-A (see FIG. 9);

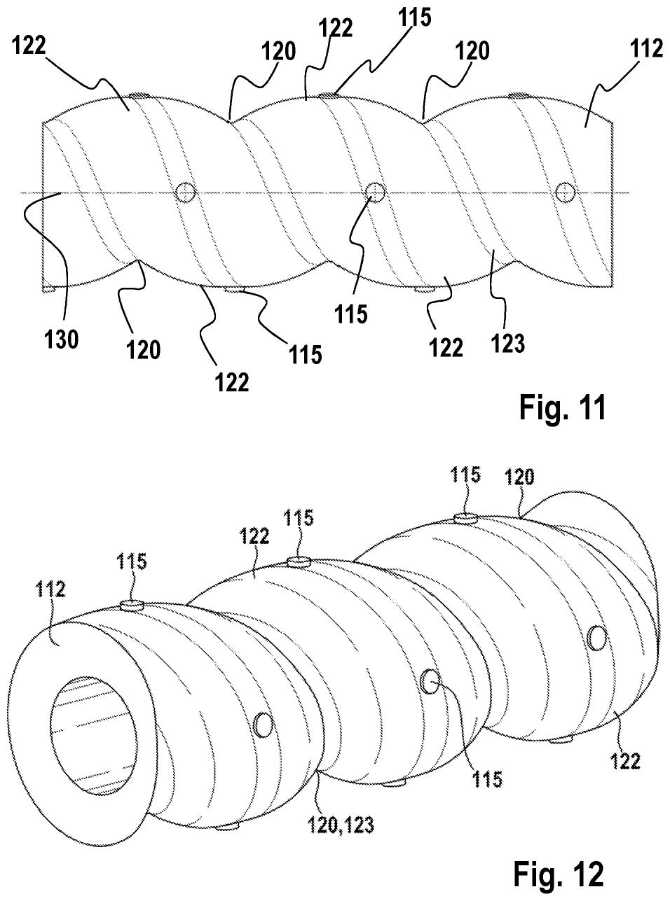

FIG. 11 shows a fifth exemplary embodiment in a second, more strongly inflated state in a view from the side; and

FIG. 12 shows the exemplary embodiment according to FIG. 11 in the state shown in FIG. 11, in a perspective view from the side.

The first exemplary embodiment of a device according to the invention shown in FIGS. 1, 2 and 6 and 7 comprises a catheter shaft, having a distal end denoted by reference numeral 10 in FIG. 1. The device furthermore comprises a balloon 12, which is shown partially inflated in FIGS. 1 and 6. The balloon 12 can be fastened to the preferably dual lumen shaft, which forms the distal end 10, or a shaft separate therefrom. The balloon 12 is furthermore connected to a gas source or a liquid source (NaCl solution) for inflation.

The balloon can be detachably fastened to the respective shaft. In this way, the device according to the invention can be adapted to the diameter of the respective vessel to be treated by replacing the balloon.

Moreover, electrodes 15 are disposed at the distal end 10 of the catheter shaft, which are preferably designed as annular electrodes.

FIGS. 2 and 7 shows a more strongly inflated stated compared to FIG. 1. In the rhombus-shaped first regions 20, in which fibers are embedded in the balloon envelope having lower elasticity than the balloon envelope in the remaining, second regions 22, rhombus-shaped constrictions are formed, which are recessed, while the balloon envelope protrudes further in the second regions 22 in a protuberance- or pillow-like manner. The distal end 10 of the catheter shaft comprising the electrodes 15 is disposed in the first region 20.

The differing extensibilities in the first regions 20 and in the second regions 22 can, alternatively, also be achieved by using differing materials in these regions. For example, the balloon envelope can be made of PI in the first regions 20, and of PU in the second regions 22.

In the inflated state, the differing extensibilities of the regions 20, 22 create a difference in height h (see FIG. 2) between the second regions 22 and the first regions 20, which is at least 0.5 mm, and preferably ranges between 1 mm and 2.5 mm. The difference in height is measured in the radial direction based on the balloon envelope.

In the second exemplary embodiment illustrated in FIGS. 3, 4 and 8, fibers 23 having the lower elasticity, and thus the first regions 20, extend parallel to the longitudinal axis 30 of the balloon 12. Since the elasticity of the balloon envelope is less in the first regions 20 than in the second regions 22, a cross-sectional shape of the balloon that resembles a butterfly is created (see FIG. 4). The distal end 10 of the catheter shaft comprising the electrodes 15 is disposed in the grooves or constrictions, which form the first regions 20 in the inflated state.

In the third exemplary embodiment shown in FIG. 5, the fibers 23, which are embedded into the balloon envelope in the first region 20 and have lesser elasticity, extend around the longitudinal axis 30 of the balloon 12 in a helical manner. The distal end 10 of the catheter comprising the electrodes 15 extends parallel to these fibers and, in the inflated state, is disposed in the corresponding grooves of the first region 20.

By way of the above-described exemplary embodiments of a device according to the invention, it is possible to achieve secure positioning of the electrodes 15 of the catheter on the inside wall of the treated vessel with good wall contact. Additionally, the first regions 20 receding in the inflated state create clearances, so that the blood can continue to flow in the vessel and thereby cool the treated site on the vessel.

In the exemplary embodiments of a device according to the invention shown in FIGS. 9 to 12, the electrodes 115 are disposed on the outside of the envelope of the balloon 112. The electrical connection of the electrically active electrodes 115 to a voltage source or further electronic components for measuring potentials or for ablation is not shown.

The exemplary embodiment shown in FIGS. 9 and 10 resembles that of FIGS. 3, 4 and 8, wherein the electrodes are disposed on the outside of the envelope of the balloon 112 in the protruding second regions 122. The elasticity of the balloon 112 in the receding first regions 120 is reduced by the non-extensible fiber 123 in the direction of the longitudinal axis 130 of the balloon 112. In the second regions 122, the balloon 112 additionally comprises sections 135 having a reduced wall thickness (see FIG. 10). In this way, an electrode 115 disposed in this section 135 protrudes more strongly upon inflation and is thus given even better contact with the vascular wall.

In the balloon 112, furthermore a tube 137 for feeding the inflation medium is disposed. Moreover, a clamping ring 140 is provided at the proximal end of the balloon 112, which is used to fix and support the balloon 112, and in particular the non-extensible fibers 123 integrated into the envelope of the balloon 112, on the tube 137. The clamping ring 140 is disposed on the outside of the balloon 112. Such a design comprising a tube for feeding the inflation medium and a clamping ring is also conceivable for the exemplary embodiment shown in FIGS. 3, 4 and 8.

In the exemplary embodiment according to FIGS. 9 and 10, furthermore a catheter, which is electrically inactive or likewise electrically active, can be fixed and guided past the treatment site and be used there to measure electrical potentials, pressures or temperatures, for example, or likewise for ablation, and for guiding guide wires, for example, and for applying liquids or other media.

Finally, FIGS. 11 and 12 show a further exemplary embodiment in which the balloon is designed analogously to the balloon of the exemplary embodiment according to FIG. 5 in terms of the elasticity thereof. The balloon 112 comprises fibers 123 integrated into the envelope, which have only little elasticity and extend about the longitudinal axis 130 of the balloon 112 in the form of a helix. The electrodes 115, which make good contact with the treated vessel due to the high extension in the second regions 122, are disposed on the outside of the balloon 112 in the second regions 122. Due to the irregular shape of the balloon 112, clearances are created in the receding first regions 120 in which the fibers 123 extend, which allow the blood to continue to flow in the vessel and thereby cool the treated site on the vessel.

### LIST OF REFERENCE NUMERALS

10 distal end of the catheter shaft
12 balloon
15 electrode
20 first region
22 second region
23 fiber
30 longitudinal axis of the balloon 12
112 balloon
115 electrode
120 first region
122 second region
123 fiber
130 longitudinal axis of the balloon 112
135 section
137 pipe
140 clamping ring

1-11. (canceled)

12. A device for delivering energy and/or measuring electrical activity in a tubular part of a body, the device comprising:
a catheter, an inflatable balloon and at least one electrode;
a distal end of said catheter with said at least one electrode being disposed on an outside of an envelope of said balloon;
at least one first region of the envelope of said balloon having an elasticity being less than an elasticity of at least one second region that is different from said first region; and
said distal end of said catheter with said at least one electrode being disposed in said at least one first region.

13. The device according to claim 12, wherein the envelope of said balloon comprises fibers in said at least one first region which limit the elasticity of the envelope of said balloon in said at least first region.

14. The device according to claim 13, wherein said fibers of said first region comprise one or more materials selected from the group consisting of PEEK, Kevlar, polyimide, polyamide and metals.

15. The device according to claim 12, wherein the envelope of said balloon has a greater wall thickness in said at least one first region than in said at least one second region.

16. The device according to claim 12, wherein said at least one first region has a rhombus shape.

17. The device according to claim 12, wherein said at least one first region extends in a direction of a longitudinal axis of said balloon.

18. The device according to claim 12, wherein said at least one first region extends helically in a spiral shape about a longitudinal axis of said balloon.

19. The device according to claim 12, wherein said distal end of said catheter is fixed in points on the envelope of said balloon.

20. The device according to claim 12, wherein said at least one electrode is a conductive ring or a conductive half shell.

21. The device according to claim 12, wherein said at least one electrode is disposed in or on the envelope of said balloon in said at least one second region.

22. A device for delivering energy and/or measuring electrical activity in a tubular part of a body, the device comprising:
an inflatable balloon and at least one electrode;
said at least one electrode being disposed on an outside of an envelope of said balloon;
at least one first region of the envelope of said balloon having an elasticity being less than an elasticity of at least one second region that is different from said first region; and
said at least one electrode being disposed in said at least one second region.

23. The device according to claim 22, wherein the envelope of said balloon comprises fibers in said at least one first region which limit the elasticity of the envelope of said balloon in said at least first region.

24. The device according to claim 23, wherein said fibers of said first region comprise one or more materials selected from the group consisting of PEEK, Kevlar, polyimide, polyamide and metals.

25. The device according to claim 22, wherein the envelope of said balloon has a greater wall thickness in said at least one first region than in said at least one second region.

26. The device according to claim 22, wherein said at least one first region has a rhombus shape.

27. The device according to claim 22, wherein said at least one first region extends in a direction of a longitudinal axis of said balloon.

28. The device according to claim 22, wherein said at least one first region extends helically in a spiral shape about a longitudinal axis of said balloon.

29. The device according to claim 22, wherein a distal end of said catheter is fixed in points on the envelope of said balloon.
